(19) United States
(12) Patent Application Publication
(10) Pub. No.: US 2003/0232409 A1
(43) Pub. Date: Dec. 18, 2003

(54) Immortal porcine cells

(75) Inventors: James A. Farris, Lino Lakes, MN (US); Douglas N. Foster, Hudson, WI (US); Scott M. O'Grady, White Bear Lake, MN (US)

Correspondence Address: MUETING, RAASCH & GEBHARDT, P.A., P.O. BOX 581415, MINNEAPOLIS, MN, 55458, US

(73) Assignee: Regents of the University of Minnesota, Minneapolis, MN

(21) Appl. No.: 10/385,882

(22) Filed: Mar. 11, 2003

Related U.S. Application Data
(60) Provisional application No. 60/363,129, filed on Mar. 11, 2002.

(51) Int. Cl. C12P 21/02; C12N 5/06; C12N 9/22

(52) U.S. Cl. 435/69.1; 435/199; 435/320.1; 435/325

(57) ABSTRACT

The present invention provides immortal porcine cells. The cells contain a polynucleotide encoding an exogenous telomerase reverse transcriptase polypeptide. Also provided are methods of making and using immortal porcine cells.

### CONTINUING APPLICATION DATA

This application claims the benefit of U.S. Provisional Application Serial No. 60/363,129, filed Mar. 11, 2002, which is incorporated by reference herein.

### BACKGROUND

Mammalian somatic cells normally exhibit limited growth potential and senescence after a defined number of cell divisions. This is due, in part, to the progressive shortening of telomeres that occurs during DNA replication. Telomers are the distal ends of eukaryotic chromosomes composed of noncoding, tandemly repeated DNA sequences that vary from one species to another. In humans and other vertebrates, the repeat unit is (TTAGGG)ₙ in the 5′ to 3′ direction. The progressive shortening of the telomeres in normal somatic cells is a problem with end replication on the lagging strand. To replicate the lagging strand, DNA polymerization starts from several RNA primers, which are elongated to create DNA fragments termed Okazaki fragments. Removal of the terminal RNA primer on the lagging strand leaves a gap that ordinarily is filled by extensions of the next Okazaki fragment. Since there is no template for the last Okazaki fragment beyond the 5′ end of the chromosome, one strand cannot be synthesized to its very end. This end replication problem predicts the progressive reductions of chromosomal DNA at the 3′ ends during multiple cell cycles (Dahse et al., Clinical Chemistry, 43, 708-714 (1997)). Telomerase corrects the loss of genomic sequences at each replication cycle by the addition of terminal sequences. In humans, this ribonucleoprotein complex includes at least three subunits—hTERT (human telomerase reverse transcriptase), hTR (human telomerase RNA) and TP1 (telomerase-associated protein). Telomerase activity has been observed in most malignant tumors and germ cells, but not usually in normal somatic cells I (Meyerson, J. Clinical Oncology, 18, 2626-2634 (2000)). In recent years, however, telomerase activity has been shown in normal human endometrial glandular epithelial cells, correlating with the proliferative phase of the menstrual cycle (Tanaka et al., Am. J. Pathology, 153, 1985-1991 (1998)). hTR and TP1 were shown to be constitutively expressed in normal endometrial tissues, while hTERT was shown to be the limiting factor for telomerase activity (Kyo et al., Int. J. Cancer, 80, 60-63 (1999)).

### SUMMARY OF THE INVENTION

The present invention provides immortalized porcine cells containing a non-porcine telomerase reverse transcriptase. The ability of human telomerase reverse transcriptase to make these cells immortal was unexpected. Telomerase reverse transcriptase is only one of at least three heterologous molecules that interact to form telomerase and, based on comparisons of the amino acid sequences of the known telomerase reverse transcriptases (for instance, human, mouse, and african clawed toad) and work with the expression of human telomerase reverse transcriptase in cells of other species, it was expected that a human telomerase reverse transcriptase would not be able to interact with the other porcine subunits to produce an complex having telomerase activity.

Accordingly, the present invention provides an immortal cell that includes a polynucleotide encoding an exogenous telomerase reverse transcriptase polypeptide. The complement of the polynucleotide hybridizes to nucleotides 57 to 3455 of SEQ ID NO:1 in a solution containing 2×SSC and 50% formamide at 42° C. for at least about 18 hours, followed by 3 washes for about 30 minutes each at about 50° C. to about 65° C. in a solution containing 0.1×SSC and 0.1% SDS. In another aspect, the immortal cell of the present invention includes a polynucleotide encoding an exogenous telomerase reverse transcriptase polypeptide, wherein the polynucleotide has at least about 80% identity to nucleotides 57 to 3455 of SEQ ID NO:1. In yet another aspect, the immortal cell includes a polynucleotide encoding an exogenous telomerase reverse transcriptase polypeptide having the amino acid sequence of SEQ ID NO:2.

The polynucleotide may be isolated from a human, include nucleotides 57 to 3455 of SEQ ID NO:1, and/or encode a polypeptide of SEQ ID NO:2. The polynucleotide may also be integrated into the cell genome.

The immortal cell may be diploid or aneuploid. The immortal cell may be obtained from uterine endometrial glandular tissue, and optionally, an epithelial cell. The immortal cell may contain a polypeptide, for instance cFOS, cyclin D1, or p53, at a greater concentration compared to a primary cell incubated under the same conditions.

In another aspect, the immortal cell of the present invention includes an exogenous telomerase reverse transcriptase polypeptide, wherein the polypeptide has at least about 80% identity to SEQ ID NO:2. The polypeptide may have the amino acid sequence of SEQ ID NO:2. The immortal cell may be diploid or aneuploid. The immortal cell may be obtained from uterine endometrial glandular tissue. The cell may be, for instance, an epithelial cell. The immortal cell may contain a polypeptide, for instance cFOS, cyclin D1, or p53, at a greater concentration compared to a primary cell incubated under the same conditions.

The present invention further provides a method for making an immortal cell. The method includes providing an isolated primary porcine cell, preferably an cell obtained from uterine endometrial glandular tissue, and introducing to the cell a polynucleotide encoding an exogenous telomerase reverse transcriptase polypeptide. The method also includes identifying a cell that contains the polynucleotide, and replicating the cell containing the polynucleotide in culture at least about 65 passages.

Also provided by the present invention is a method for measuring an amount of a virus in a sample. The method includes providing a serial dilution of sample that includes a virus, contacting an immortal porcine cell of the present invention with a portion of the serial dilution, and measuring the amount of virus present in the virus dilution. Preferably, the virus is a a porcine reproductive and respiratory syndrome virus.

Further provided by the present invention is a method for producing virus. The method includes providing an immortal porcine cell that contains an exogenous telomerase reverse transcriptase polypeptide, introducing a virus to the immortal porcine cell, incubating the immortal porcine cell, and harvesting the virus produced by the immortalized porcine cell. Preferably, the virus is a porcine reproductive and respiratory syndrome virus.

The present invention also provides a method of evaluating toxicity of a compound. The method includes contacting an immortal porcine cell of the present invention with a compound, and incubating the immortal porcine cell, wherein an increase in doubling time of the immortal porcine cell indicates the compound is toxic.

The present invention further provides an immortal porcine cell having the designation PEGE1.

Unless otherwise specified, “a,” “an,” “the,” and “at least one” are used interchangeably and mean one or more than one.

### BRIEF DESCRIPTION OF THE FIGURES

FIG. 1. A schematic illustration of a human metaphase chromosome. The distal ends, or telomeres, are composed of the DNA repeats, TTAGGG (5′ to 3′ direction), stabilizing and protecting the chromosome from enzymatic end degradation. Telomerase counteracts the loss of genomic sequences at each replication cycle by the addition of terminal sequences.

FIG. 2. Population doubling (PDL) as a function of passage. The population doubling time shows a significant increase with increasing passage number.

FIG. 3. A representative trace illustrating the PGE2-stimulated current with (A) primary cells, passage 4; (B) immortalized cells, passage 19, and (C) passage 44-51. 10 μM indomethecin, 5 μM benzamil, 3 μM PGE2, and 100 μM NPPB was used in each experiment. In A, B and C, n=5, 6 and 5, respectively.

FIG. 4. A representative trace illustrating the 8 cpt cAMP-stimulated current with (A) primary cells, passage 4; (B) immortalized cells, passage 19, and (C) passage 44-51. 10 μM indomethecin, 5 μM benzamil, 50 μM 8 cpt cAMP, and 100 μM NPPB was used in each experiment. In A, B and C, n=6 for each.

FIG. 5. Mean basal Isc, potential difference and resistance comparing primary cells (passage 4), and immortalized cells (passage 19 and 44-51). A. Mean basal Isc for p4, p19 and p44-51 is 9.4 μA+0.8, 12.3 μA+1.7 and 4.0 μA+0.5 (n=12, 15 and 12), respectively. B. Mean basal potential difference for p4, p19 and p44-51 is 4.2 mV+0.5, 6.2 mV+0.9 and 1.3 mV+0.2 (n=12, 15 and 15), respectively. C. Mean resistance for p4, p19 and p44-51 is 3238.5 Ωcm2+85.9, 3410.2 Ωcm2+85.9 and 6506.0 Ωcm2+500.0 (n=12, 12 and 9), respectively.

FIG. 6. Mean stimulated and inhibitory Isc comparing primary cells (passage 4), and immortalized cells (passage 19 and 44-51). A. Mean indomethecin-sensitive Isc for p4, p19 and p44-51 is 3.4 μA+0.5, 5.0 μA+0.8 and 0.6 μA+0.1 (n=12, 15 and 14), respectively. Mean benzamil-sensitive Isc for p4, p19 and p44-51 is 0.5 μA+0.1, 1.6 μA+0.3 and 1.7 μA+0.3 (n=12, 15 and 15), respectively. B. Mean PGE2-stimulated Isc for p4, p19 and p44-51 is 40.3 μA+2.1, 69.0 μA+7.8 and 35.0 μA+2.0 (n=5, 6 and 5), respectively. Mean 8 cpt cAMP-stimulated Isc for p4, p19 and p44-51 is 25.9 μA+1.8, 33.2 μA+1.0 and 15.6 μA+0.8 (n=6 for each), respectively. C. Mean PGE2-stimulated NPPB-sensitive Isc for p4, p19 and p44-51 is 24.9 μA+1.8, 33.4 μA+1.8 and 12.5 μA+2.2 (n=5, 6 and 5), respectively. Mean 8 cpt cAMP-stimulated NPPB-sensitive Isc for p4, p19 and p44-51 is 17.6 μA+1.5, 24.3 μA+0.8 and 14.5 μA+1.1 (n=6 for each), respectively.

FIG. 7. Western blot analysis showing changes in expression for cFOS, p53, cMyc, cyclin D1 and E2F in primary and immortalized PEG cells.

FIG. 8. Schematic diagram showing key regulatory proteins that control cell cycle progression in eukaryotic cells. Note that p53 exerts its inhibitory actions through activation of p21. Activation of CDK4 by cyclin D1 leads to phosphorylation of Rb, resulting in liberation of E2F and subsequent upregulation of downstream cyclins involved in cell cycle progression.

FIG. 9. A. Nucleotide sequence (SEQ ID NO:1) encoding a telomerase reverse transcriptase and B. amino acid sequence (SEQ ID NO:2) encoded by the nucleotides 57 to 3455.

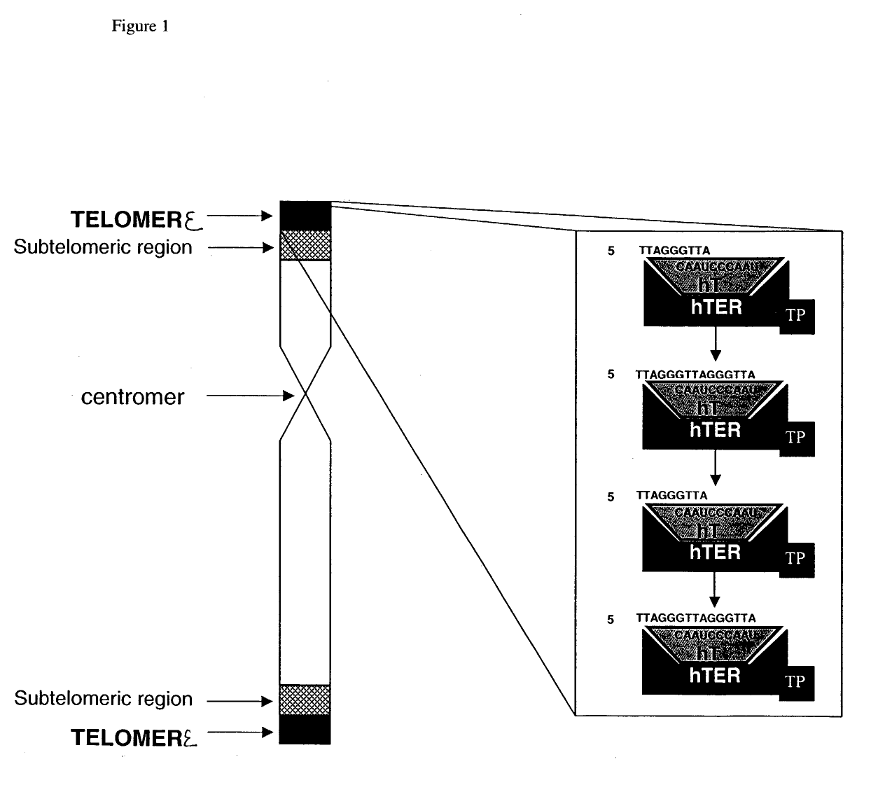

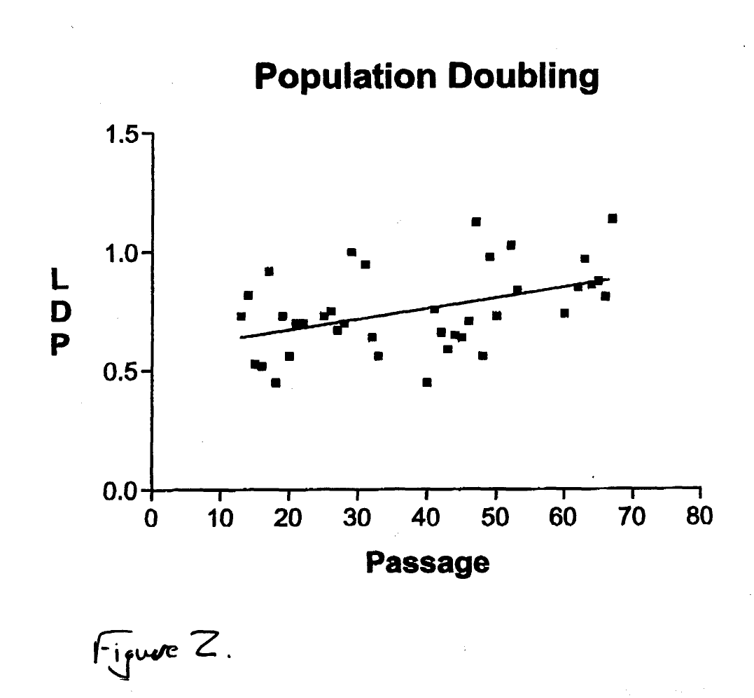

### DETAILED DESCRIPTION OF PREFERRED EMBODIMENTS OF THE INVENTION

The present invention provides immortal porcine cells. “Porcine” refers to an animal that is a member of the family Suidae. Preferably, the animal is Sus scrofa. Porcine cells are typically able to grow in culture for no more than about 15 to about 30 passages. A passage is the splitting of a confluent population of cells about 1:2 or about 1:4. A passage generally allows cells to undergo about 2 to about 4 doublings, preferably about 3, before another split is performed. An immortal porcine cell of the present invention is able to grow in culture for at least about 65 passages, preferably, at least about 80 passages, more preferably, at least about 100 passages after isolation of the cell from an animal. The immortal cells typically maintain a doubling time of from about 0.5 per day to about 1.1 per day, preferably, about 0.8 per day. Preferably, the immortalized cells are not transformed. Unlike transformed cells, immortalized cells are density dependent (e.g., contact inhibited). Furthermore, immortalized cells do not have the neoplastic characteristics typically displayed by transformed cells. Such neoplastic characteristics include the ability to grow in soft agar, and the ability to form tumors when injected into nude and/or SCID mice, or an animal that is a member of the family Suidae.

The immortal porcine cells of some aspects of the present invention include an exogenous telomerase reverse transcriptase (TERT) polypeptide. As used herein, an exogenous polynucleotide is one that has been introduced to a cell, and is not naturally present in the cell. The polynucleotide is typically introduced as part of a vector. An exogenous polypeptide is a polypeptide that is encoded by an exogenous polynucleotide, and is not naturally present in the cell. Methods of making a porcine cell containing an exogenous telomerase reverse transcriptase polypeptide are described herein.

As used herein, “polypeptide” refers to a polymer of amino acids linked by peptide bonds and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations and the like. A polypeptide can be obtained directly from a human, or can be prepared with the aid of recombinant, enzymatic, or chemical techniques. As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. Coding sequence, non-coding sequence, and regulatory sequence are defined below. A polynucleotide can be obtained directly from a human, or can be prepared with the aid of recombinant, enzymatic, or chemical techniques. A polynucleotide can be linear or circular in topology. A polynucleotide can be, for example, a portion of a vector, such as an expression or cloning vector, or a fragment. An “isolated” polypeptide or polynucleotide means a polypeptide or polynucleotide that has been either removed from its natural environment, produced using recombinant techniques, or chemically or enzymatically synthesized.

As used herein, a “coding sequence,” also referred to herein as a coding region, refers to a polynucleotide that encodes an mRNA and, when placed under the control of appropriate regulatory sequences, expresses the encoded RNA. An mRNA can be translated in the host cell to yield a biologically active polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

A preferred example of a telomerase reverse transcriptase polypeptide has the amino acid sequence present at SEQ ID NO:2 (GenBank Accession number AAC51724). Additional examples of telomerase reverse transcriptase polypeptides include those having similarity with the amino acid sequence of SEQ ID NO:2. The similarity is referred to as structural similarity and is generally determined by aligning the residues of the two amino acid sequences (i.e., a candidate amino acid sequence and the amino acid sequence of SEQ ID NO:2) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence is the amino acid sequence being compared to an amino acid sequence present in SEQ ID NO:2. A candidate amino acid sequence can be isolated from a human, or can be produced using recombinant techniques, or chemically or enzymatically synthesized. Preferably, two amino acid sequences are compared using the standard protein-protein Blast (Blastp) search algorithm, a version of which is available on the world wide web (www) at ncbi.nlm.nih.gov/BLAST/. Preferably, the default values for all Blastp search parameters are used, including composition-based statistics on, low complexity filter on, expect=10, word size=3, matrix=BLOSUM62, and gap costs: existence=11 and extension=1. In the comparison of two amino acid sequences using the Blastp search algorithm, structural similarity is referred to as “identities.” Preferably, a candidate amino acid sequence has, in increasing order of preference, at least about 80%, at least about 90%, at least about 95%, most preferably, at least about 98% identity to SEQ ID NO:2. A polypeptide having structural similarity to of SEQ ID NO:2 has telomerase reverse transcriptase activity. Whether such a polypeptide has activity can be determined as described herein.

In other aspects of the present invention, the immortal porcine cells contain a polynucleotide encoding an exogenous telomerase reverse transcriptase polypeptide. A preferred polynucleotide contains a nucleotide sequence encoding the polypeptide having the amino acid sequence of SEQ ID NO:2. An example of the class of nucleotide sequences encoding such a polypeptide is the coding sequence present at nucleotides 57 to 3455 of SEQ ID NO:1 (GenBank Accession number AF018167). This class of nucleotide sequences is large but finite, and the nucleotide sequence of each member of the class can be readily determined by one skilled in the art by reference to the standard genetic code.

Additional examples of polynucleotides encoding a telomerase reverse transcriptase polypeptide include those having similarity with nucleotides 57 to 3455 of SEQ ID NO:1. The similarity is referred to as structural similarity and is determined by aligning the residues of the two polynucleotides (i.e., the nucleotide sequence of the candidate coding region and the nucleotide sequence of the coding region of SEQ ID NO:1) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate coding region is the coding region being compared to a coding region present in SEQ ID NO:1 (i.e., nucleotides 57 to 3455 of SEQ ID NO:1). A candidate nucleotide sequence can be isolated from a human, or can be produced using recombinant techniques, or chemically or enzymatically synthesized. Preferably, two nucleotide sequences are compared using the standard nucleotide-nucleotide Blast (Blastn) search algorithm, a version of which is available on the world wide web (www) at ncbi.nlm.nih.gov/BLAST/. Preferably, the default values for all Blastn search parameters are used, including low complexity filter on, expect=10, and word size=11. In the comparison of two nucleotide sequences using the Blastn search algorithm, structural similarity is referred to as “identities.” Preferably, a polynucleotide includes a nucleotide sequence having a structural similarity with the coding region of SEQ ID NO:1 of at least about 80%, more preferably at least about 90%, most preferably at least about 95% identity. A polypeptide encoded by a polynucleotide having structural similarity to nucleotides 57 to 3455 of SEQ ID NO:1 has telomerase reverse transcriptase activity. Whether such a polypeptide has activity can be determined as described herein.

Examples of polynucleotides encoding a telomerase reverse transcriptase polypeptide also include those having a complement that hybridizes to nucleotides 57 to 3455 of SEQ ID NO:1 under conditions of high stringency. The term “complement” refers to the ability of two single stranded polynucleotides to base pair with each other, where an adenine on one polynucleotide will base pair to a thymine on a second polynucleotide and a cytosine on one polynucleotide will base pair to a guanine on a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. As used herein, “hybridizes,” “hybridizing,” and “hybridization” means that a single stranded polynucleotide forms a noncovalent interaction with a complementary polynucleotide under certain conditions. Typically, one of the polynucleotides is immobilized on a membrane. A polypeptide encoded by a polynucleotide having a complement that hybridizes to nucleotides 57 to 3455 of SEQ ID NO:1 has telomerase reverse transcriptase activity. Whether such a polypeptide has activity can be determined as described herein.

High stringency hybridization conditions can be determined using methods routine and known in the art. Preferably, high stringency conditions use hybridization buffer (2×SSC [where 1×SSC is 150 mM NaCl, and 15 mM sodium citrate] and 50% formamide) containing a labeled polynucleotide. Hybridization is allowed to occur at about 42° C. for at least about 18 hours. The membrane is washed under high stringency conditions at about 50° C. to about 65° C., preferably about 55° C. to about 60° C., 3 times for about 30 minutes each in a solution containing 0.1×SSC and 0.1% SDS. Preferably, a probe will hybridize to nucleotides 57 to 3455 of SEQ ID NO:1 under the high stringency conditions. Preferably, at least about 20 nucleotides of the complement hybridize with nucleotides 57-3455 of SEQ ID NO:1, more preferably at least about 50 nucleotides, most preferably at least about 100 nucleotides.

Telomerase reverse transcriptase is one of at least three heterologous molecules that interact to form telomerase, and telomerase reverase transcriptase has an enzymatic activity when it is complexed with the other subunits of telomerase. Thus, the telomerase reverse transcriptase activity of a polypeptide can be tested indirectly by assaying for telomerase activity. Mortal cells typically have low or undetectable telomerase activity, but the presence of an exogenous telomerase reverse transcriptase polypeptide into such cells often results in telomerase activity. To assay for telomerase reverse transcriptase activity, a cell containing an exogenous telomerase reverse transcriptase polypeptide is cultured, and the telomerase activity of the resulting cell is determined. When testing whether a polypeptide having structural similarity to SEQ ID NO:2, encoded by a polynucleotide having structural similarity to nucleotides 57 to 3455 of SEQ ID NO:1, or encoded by a polynucleotide having a complement that hybridizes to nucleotides 57 to 3455 of SEQ ID NO:1 has telomerase reverse transcriptase activity, the cell can be a human cell or a porcine cell, preferably, a porcine cell, that has low or undetectable levels of telomerase activity without the exogenous telomerase reverse transcriptase. Preferably, the cells is a differentiated somatic cell that is mortal and non-transformed.

Assays that measure the telomerase activity of a cell are routine and known in the art. A preferred assay includes the telomeric repeat amplification protocol (see, for instance, U.S. Pat. No. 5,629,154 (Kim et al.), U.S. Pat. No. 5,837,453 (Harley et al.), and U.S. Pat. No. 5,863,726 (Harley et al.)). For instance, about 1×10⁵ to about 1×10⁶ cells can be pelleted and suspended about 200 microliters (μl) of CHAPS buffer (a 1× solution is 10 mM Tris-HCl, pH 7.5, 1 mM MgCl₂, 1 mM EGTA, 0.1 mM benzamidine, 5 mM β-mercaptoethanol, 0.5% CHAPS, and 10% glycerol). After incubation on ice for about 30 minutes, the lysate is centrifuged at about 12,000×g for about 20 minutes at about 4° C. After transfer of the supernatant to a new tube, the protein concentration of the supernatant is determined. Methods for measuring protein concentration are routine and known in the art. About 1.5 micrograms (μg) of protein or less is used in each assay.

Each assay includes the following: about 20 mM Tris-HCl (pH about 8.3), about 1.5 mM MgCl₂, about 63 mM KCl, about 0.005% Tween 20, about 1 mM EGTA, about 2 Units Taq polymerase, dNTPs, a forward primer (for instance, AATCCGTCGAGCAGAGTT (SEQ ID NO:3), a reverse primer (for instance, GCGCGGCTAACCCTAACCCTAACC (SEQ ID NO:4), or GCGCGGCTTACCCTTACCCTTACCCTAACC (SEQ ID NO:5)), and protein from the cell lysate. This mixture is subjected to a 2-step PCR of about 30 to about 33 cycles of the following: about 94° C. for about 30 seconds, and about 59° C. for about 30 seconds. A portion of this reaction mixture can be resolved, typically on a gel, that permits the resolution of DNA fragments of at least about 50 base pairs. Negative controls that may be used include heat inactivation (for instance, incubation at about 85° C. for about 10) of a sample of the lysate prior to use in the assay. Positive controls include immortal cells (for instance, Hela cells) and germ cells (for instance, porcine germ cells). If the cell used to create the lysate have telomerase activity, a ladder of amplified DNA fragments will be present starting at 50 bases and increasing in 6 base increments. Thus, the presence of such a ladder indicates that the cell has telomerase reverse transcriptase activity. Reagents sufficient to perform telomerase activity assays are commercially available from Intergen Company (Norcross, Ga.), under the trade name TRAPEZE telomerase detection kit.

An immortal porcine cell of the present invention may contain some polypeptides at higher concentrations than mortal (e.g., primary) porcine cells. Such polypeptides include the cell cycle regulatory polypeptides cFOS, cyclin D1, and p53. The relative concentrations of these polypeptides in immortal porcine cells and mortal porcine cells can be determined using methods routine to a person of skill in the art. The ploidy of an immortal porcine cell of the present invention may vary, including diploid or aneupoloid (for instance, tetraploid, hyperploid, or hypoploid). A normal cell of Sus scrofa has 20 chromosomes (i.e., 1N is 20). Methods for determining the ploidy of a cell are routine and know to the art.

A preferred immortal porcine cell has the designation PEGE1. This cell is an epithelial cell obtained from an endometrial gland, has telomerase activity, and is contact inhibited. This cell has been in culture for at least about 150 passages. This cell contains the polypeptides cFOS, cyclin D1, and p53 at concentrations that are greater than mortal porcine endometrial cells.

The present invention also provides methods for making the immortal porcine cells described herein. The method includes providing an isolated primary porcine cell, and introducing to the cell a polynucleotide encoding a telomerase reverse transcriptase polypeptide. An “isolated” cell is a cell that has been physically separated from other cells to which it is attached in its natural environment. As used herein a “primary” cell refers to a cell that has been recently isolated from intact tissues. When the primary cell is a porcine cell, it is typically mortal, and grows in culture for no more than about 15 to about 30 passages. Primary cells also have low or undetectable telomerase activity when assayed as described herein. The method further includes identifying a cell that contains the polynucleotide, and replicating the cell in culture for at least about 65 passages. Preferably, the cell containing the polynucleotide has telomerase activity.

Primary cells that can be used to make an immortal porcine cell of the present invention may be obtained from an animal of the family Suidae, preferably a Sus scrofa. The cells can be from different organs, including, for instance, skin, lung, pancreas, liver, stomach, intestine, heart, bladder, kidney, and uterus. Preferably, the cells are obtained from uterine endometrial glandular tissues. The animal may be an embryo or an adult.

An immortal porcine cell of the present invention is not limited by the cell type. For instance, the immortal porcine cell can be an epithelial cell, an epidermal cell, a keratinocyte, a melanocyte, a chondrocyte, a fibroblast, a cardiac muscle cells, or other muscle cells. Preferably, the cell is an epithelial cell. In some aspects of the invention, the cells of the present invention do not have viral-encoded reverse transcriptase activity. In some aspects, the cells of the present invention do not contain a porcine reproductive and respiratory syndrome virus. Methods for measuring reverse transcriptase and whether a cell contains a porcine reproductive and respiratory syndrome virus are known to the art.

The polynucleotide encoding the telomerase reverse transcriptase polypeptide is typically present on a vector. A vector is a replicating polynucleotide to which another polynucleotide may be attached so as to bring about the replication of the attached polynucleotide. Construction of vectors containing a polynucleotide of the invention employs standard ligation techniques known in the art (see, e.g., Sambrook et al, Molecular Cloning: A Laboratory Manual., Cold Spring Harbor Laboratory Press (1989)). A vector can provide for further cloning (amplification of the polynucleotide), i.e., a cloning vector, and/or the expression of the polypeptide encoded by the coding region, i.e., an expression vector. Preferably the vector is an expression vector. The term vector includes, but is not limited to, plasmid vectors, viral vectors, cosmid vectors, or artificial chromosome vectors. Preferably the vector is a plasmid.

Selection of a vector depends upon a variety of desired characteristics in the resulting construct, such as a selection marker, vector replication rate, and the like. Suitable host cells for cloning or expressing the vectors herein are prokaryote or eukaryotic cells.

An expression vector optionally includes regulatory sequences operably linked to the coding region. The invention is not limited by the use of any particular promoter, and a wide variety are known. Promoters act as regulatory signals that bind RNA polymerase in a cell to initiate transcription of a downstream (3′ direction) coding region. The promoter used in the invention can be a constitutive or an inducible promoter. Preferably, the promoter is constitutive. Preferred promoters for expression in porcine cells include SV-40 promoter, human cytomegalovirus (CMV) immediate-early enhancer/promoter region, and elongation factor 1α.

Transcription of a coding region encoding an telomerase reverse transcriptase polypeptide can be increased by inserting an enhancer sequence into the vector. Enhancers are cis-acting elements of DNA, usually having about 10 to 300 bases, that act on a promoter to increase its transcription. Enhancers are relatively orientation- and position-independent, having been found 5′ and 3′ to coding regions, within an intron as well as within the coding sequence itself. Many enhancer sequences are now known from mammalian genes (globin, elastase, albumin, alpha-fetoprotein, and insulin). Enhancers from eukaryotic cell viruses are also known and include the SV40 enhancer on the late side of the replication origin, the cytomegalovirus early promoter enhancer, the polyoma enhancer on the late side of the replication origin, and adenovirus enhancers. The enhancer may be spliced into the vector at a position 5′ or 3′ to the coding region encoding a telomerase reverse transcriptase, but is preferably located at a site 5′ of the promoter.

An expression vector can optionally include a ribosome binding site. It can also include a termination sequence to end translation. A termination sequence is typically a codon for which there exists no corresponding aminoacetyl-tRNA, thus ending polypeptide synthesis. The polynucleotide introduced to a mortal porcine cell can optionally further include a transcription termination sequence. Transcription termination sequences in vectors for eukaryotic cells typically include a polyadenylation signal 3′ of the coding region.

The polynucleotide used to transform the primary porcine cell optionally includes one or more marker sequences, which typically encode a molecule that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to ampicillin, neomycin, and formulations of phleomycin D1 including, for example, the formulation available under the trade-name ZEOCIN (Invitrogen).

A polynucleotide encoding a telomerase reverse transcriptase polypeptide can be introduced by methods routine and known in the art. Such methods include the use of DNA condensing agents, for instance, salts (e.g., calcium phosphate), cationic polymers (e.g., polylysine, polyarginine, and polyethyleneimine), and cationic lipids (e.g., DOTMA and DOTAP), or the use of electroporation. Following introduction of the polynucleotide, the cells are passaged using routine methods known in the art, and those cells able to grow in culture at least about 65 passages, preferably, at least about 80 passages, more preferably, at least about 100 passages after isolation of the cell from an animal are considered immortal. Preferably, the polynucleotide encoding a telomerase reverse transcriptase polypeptide is integrated in the genome of the cell. Optionally, the cells also have telomerase activity.

The continued culture of an immortal porcine cell of the present invention requires the use of cell culture techniques that are routine and known in the art. For instance, the cells may be passaged about twice a week by transferring cells from a confluent plate to a new plate. For instance, between about 100,000 to about 300,000 cells can be transferred to a new 10 centimeter plate.

The present invention further provides methods for producing virus. The method includes providing an immortal cell of the present invention, introducing a virus to the cell, and incubating the cell. The virus produced by the cell is then isolated. The invention is not intended to be limited by the type of viruses that may be used, and includes viruses that will replicate on porcine cells. Whether a virus replicates on porcine cells can be determined using methods routine to a person of skill in the art. A virus may also be a recombinant virus, e.g., a virus engineered using routine techniques in molecular biology. Preferred viruses include those that cause disease in animals, preferably pigs. A preferred example of a virus is the porcine reproductive and respiratory syndrome virus (see, for instance, Wensvoort et al., Vet. Q., 13, 121-130 (1991), and (Collins et al., J. Vet. Diagn. Investig., 4, 117-126 (1992)).

To produce virus, the cells of this invention can be seeded into tissue culture flasks, roller bottles, stir culture, into hollow fiber reactors or other mass culture systems. The multiplicity of infection (ratio of infectious virus particles to cells) to initiate virus stock growth will vary depending on virus strain and growth conditions. Those skilled in the art of virology and skilled in the growth of particular viruses and strains of viruses will be able to maximize virus stock yield through the standard manipulation of the multiplicity of infection, temperature, media variations, and the like, without undue experimentation. A virus made using the cells of the present invention may be used to produce compositions for immunizing an animal.

Methods for harvesting the virus after infection to obtain infectious virus stock also varies with virus strain and growth conditions. Enveloped viruses typically egress into the culture media more slowly than non-enveloped virus. Stocks of virus can be obtained from the culture media alone or from cell lysates pooled with the conditioned media. For lytic viruses (those efficient at lysing a cell during virus egress), harvesting the conditioned culture media (e.g., spent media containing virus) after a gentle centrifugation step to remove cell debris is sufficient. Again, methods for harvesting and saving virus from a wide range of virus strains are routine and known in the art.

There are a variety of methods, also all known in the art, for measuring viral growth from a culture of cells. Typically, serial dilutions of a virus stock are made and the dilutions are added to cultures of the cell of the present invention. The cells contacted with the different dilutions are incubated and then examined. Methods of evaluating the titer of a virus in the serial dilutions depend upon the characteristic pathology a virus has on cultured cells. For example, the titer of a virus producing foci of cytopathology on a cell monolayer surface are readily measured by plaque assay (as plaque forming units/ml of culture fluid or as plaque forming units/dose for vaccine inoculum virus quantification) or as tissue culture infectious dose-50 (TCID₅₀). Rapidly lytic viruses are better quantified by TCID₅₀ as the dose or dilution of virus stock capable of infecting 50% of the cultures in a defined time period.

The present invention also provides a method for producing a protein. The method includes providing an immortal porcine cell that contains an exogenous polynucleotide encoding a polypeptide other than an exogenous telomerase reverse transcriptase, and incubating the immortal porcine cell. The polypeptide produced by the immortal porcine cell is then isolated. Methods for isolating polypeptides are routine and known to the art.

The cells of the present invention may also be used in a method for evaluating the toxicity of a compound. The method includes contacting an immortal porcine cell with a compound and incubating the immortal porcine cell. The ability of the cell to function is determined. For instance, the doubling time is measured. A decrease in the ability of the cell to function, for instance, an increase in doubling time of the cell, indicates the compound is toxic.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

This Example describes the methods used to establish an immoral porcine cell line from endometrial glandular epithelial cells.

The plasmid pCI-Neo-hTERT (Counter et al., Proc. Natl. Acad. Sci. USA, 95, 14723-14728 (1998)), which encodes the human telomerase catalytic subunit, was used. The insert is a 3,450 base nucleotide sequence encoding human EST2, also known as hTERT

Epithelial cells were obtained from an endometrial gland of a sow using cell panning and centrifugation methods (see, for instance, (Deachapunya and O'Grady, J. of Physiology, 508 (Part 1), 31-47 (1998)). Cells were incubated in media made by combining 500 ml of high glucose DMEM (Invitrogen, Carlsbad, Calif., catalog number 11965-092), 5.65 ml penicillin/streptomycin (Invitrogen catalog number 04656), 5.65 ml kanamycin sulfate (Invitrogen catalog number 04643), 5.65 ml non-essential amino acids (Sigma, St. Louis, Mo., catalog number M7145), 1.13 ml bovine insulin (Sigma, catalog number T1882), and 56.5 ml fetal calf serum. The final concentration of the components in the media were 50 units/ml penicillin G, 50 units/ml streptomyscin sulfate, 100 μg/ml kanamycin sulfate, 0.1 mM non-essential amino acids, 50 μg/ml bovine insulin, and 10% serum. Cells were passaged by rinsing once in Mg⁺⁺ Ca⁺⁺ free phosphate buffered saline (PBS) for 30 seconds, removing the PBS and rinsing again in Mg⁺⁺ Ca⁺⁺ free PBS for 10 minutes. After removal of the second PBS rinse solution, a solution containing trypsin was added to the cells and incubated for 10 minutes. Loose cells were removed and transferred to fresh media containing 5% serum, and replated.

Cells were transfected with the vector by means of electroporation. Two plates of early passage 2 μg endometrial glandular epithelial (PEGE) cells were trypsinized and used for each experiment. About 6×10⁶ cells were used in a volume of 400 microliter (μl) for each electroporation reaction. The Cytomix protocol (van den Hoff et al., Nucl. Acids Res., 20, 2902 (1992)) was followed and 48 μg of the plasmid was used in each tube at various field strengths. The shorter electrical pulses worked better than longer with the best transfection occurring at 150 volts for 8 milliseconds (ms). This resulted in a field strength of approx. 375 Volts per centimeter (cm).

After transfection, the cells were plated in 10 cm dishes, and 18 hours post transfection, 900 μg per ml G418 (Calbiochem) was added to the media. At this time fibroblast as well as stromal cells populated the cell culture in minor populations compared to the epithelial cells of interest. Over the next weeks the plates were closely observed. Fibroblasts stick to the plate slightly longer than other cells when the cells are treated with trypsin, and, when added to a tissue culture plate, tend to adhere to the plate faster than other cells. By adjusting the trypsinization time and the cell sit down time, a seemingly pure population was achieved. After this selective procedure and the antibiotic selection procedure the cells increased their growth rate from 0.16 population doubling level per day (PDL/d) to >0.65 PDL/d. This increase in PDL/d occurred over the first 3 months (the first 40 passages) after isolation of the primary cells. The cells in this culture were designated PEGE1.

At greater than 65 passages, the PDL/d was 0.8+/−0.07 when passaged twice a week by transferring about 300,000 cells to a 10 cm tissue culture dish.

After growth, a density of between 2×10⁶ to 4×10⁶ cells per dish was expected, and a maximal density of 6×10⁶+/−1.2×10⁶ cells per dish was observed.

### Example 2

This Example compares primary and immortal PEGE (at passage 19 and at passages 44-51) cells functionally and morphologically. In serum containing media, the basal current (basel Isc), potential difference and resistance of primary cell monolayers at passage 4 were not significantly different from immortalized cells at passage 19. However, later passage immortalize cells did show significant differences in basal electrical properties. In addition, immortalized cells at passage 19 were similar to primary cells at passage 4 in their response to PGE2 and cAMP. Analysis of specific cell cycle regulatory proteins showed that cFOS and cyclin D1 expression was upregulated in immortalized cells compared to primary cells grown under the same conditions. The observation that early passage immortalized cells retain transport properties similar to that of primary cells indicates that these cells will be useful in future studies directed towards understanding the mechanisms and regulation of ion transport across the endometrial epithelium.

PEGE cells maintained under serum-free conditions exhibited basal, benzamil-sensitive Na+ absorption. When these cells were maintained under serum-containing conditions, they exhibited an increased basal indomethecin-sensitive current and basal anion secretion. Stimulation with PGE2 or cAMP, produced an increase in Isc that is blocked by the Cl⁻ channel blocker, 5-nitro-2-(3phenylpropylamino) benzoic acid (NPPB) (Deachapunya and O'Grady, J. of Physiology, 508 (Part 1), 31-47 (1998)). The objective of the present study was to determine whether immortalization of PEG cells using hTERT altered the transport characteristics of these cells and to identify changes in key cell cycle regulatory proteins that might have occurred as a consequence of immortalization.

Methods

PEGE primary cells, isolated and cultured as previously described (Deachapunya and O'Grady, J. of Physiology, 508 (Part 1), 31-47 (1998)), and PEGE1 cells were used. Transepithelial resistance of cell monolayers was measured using the EVOM epithelial voltohmmeter coupled to Ag/AgCl “chopstick” electrodes (World Precision Instrument, New Haven, Conn.). After 10 days, the confluent culture inserts were mounted in Ussing Chambers, bathed on both sides with standard porcine Ringer solution which was maintained at 37° C., and bubbled with 95% O₂ and 5% CO₂. Transepithelial potential difference and Isc were measured with the use of voltage-clamp circuitry from JWT Engineering (Overland Park, Kans.). The data from the voltage clamp experiments was digitized, stored and analyzed using Workbench data acquisition software (Kent Scientific Corporation, Litchfield, Conn.) and recorded with a Compaq pentium microcomputer.

Western blot analysis was performed as follows. Cell monolayers were solubilized with lysis buffer (50 millimolar (mM) Tris-HCl, 1% NP-40, 0.25% sodium deoxycholate, 150 mM NaCl, 1 mM EGTA, 1 mM PMSF, 1 microgram per milliliter (μg/ml) aprotinin and 1 mM NaF, pH 7.4) at 37° C. for 30 minutes. A protein assay was performed using a BCA Protein Assay Kit by Pierce (Rockford, Ill.). Proteins were separated by polyacrylamide gel electrophoresis (8% and 10%). Electroblotting was done using Immobilon-P (Millipore). The electroblot assembly was placed into the electroblotting apparatus (Trans-Blot Cell, Biorad) and blotting was performed at 16 Volts overnight on ice. After the blots were removed, they were washed twice then blocked in freshly prepared 1×TBS-Tween containing 3% nonfat dry milk for 30 minutes at 20° C. to 25° C. with constant agitation. After washing, blots were reacted overnight in primary antibody (anti-cFOS, anti-p53, anti-cMyc, anti-cyclin D1, and anti-E2F (all obtained from Oncogene Research Products, Boston, Mass.), in 7.5 ml or 15 ml of freshly prepared 1×TBS-tween containing 3% milk with the appropriate dilution of primary antibody. The next day, blots were washed and reacted with secondary antibody, with either goat anti-rabbit, alkaline phosphatase labeled (GAR-AP) or goat anti-mouse alkaline phosphatase labeled (GAM-AP) from Biorad. Secondary antibody was diluted 1:3000 in 1×TBS-tween containing 3% milk and reacted for approximately one hour. After washing, alkaline phosphatase color reagents were added to 100 ml 1× alkaline phosphatase color development buffer (NBT-BCIP) at room temperature. Blots were incubated in development buffer until bands were clearly developed.

All values are presented as means±SEM. n is the number of monolayers. The differences between control and treatment means were analyzed using a t-test for paired or unpaired means where appropriate. A value of p<0.05 was considered statistically significant.

Results

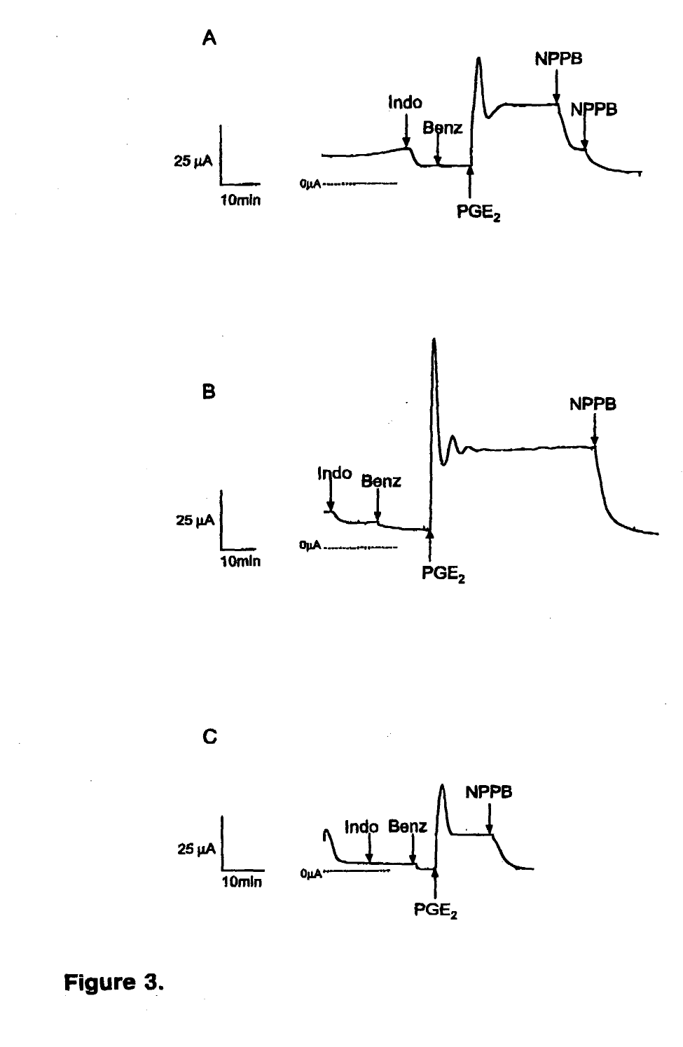

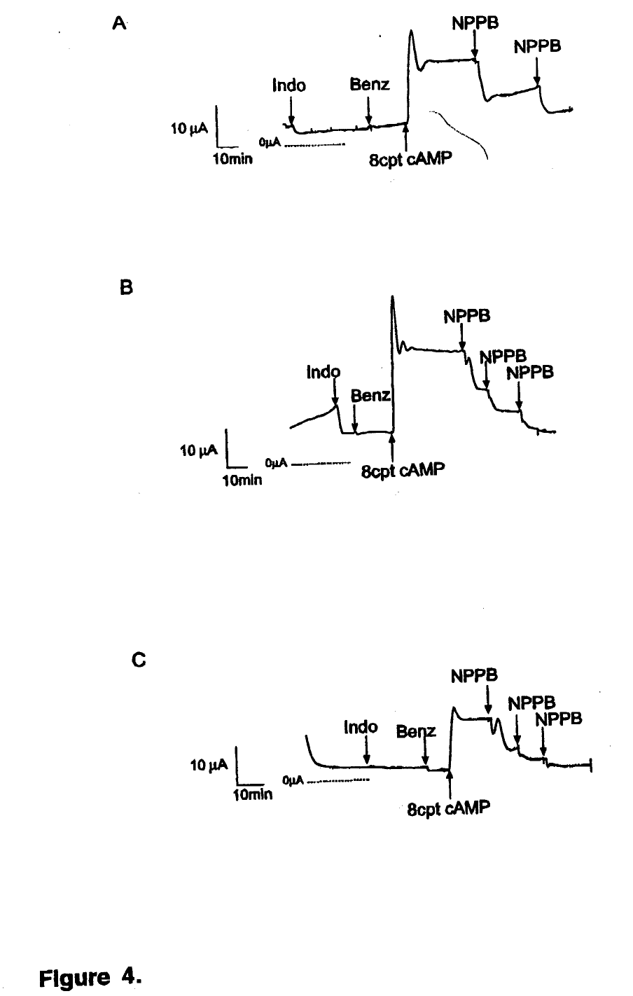

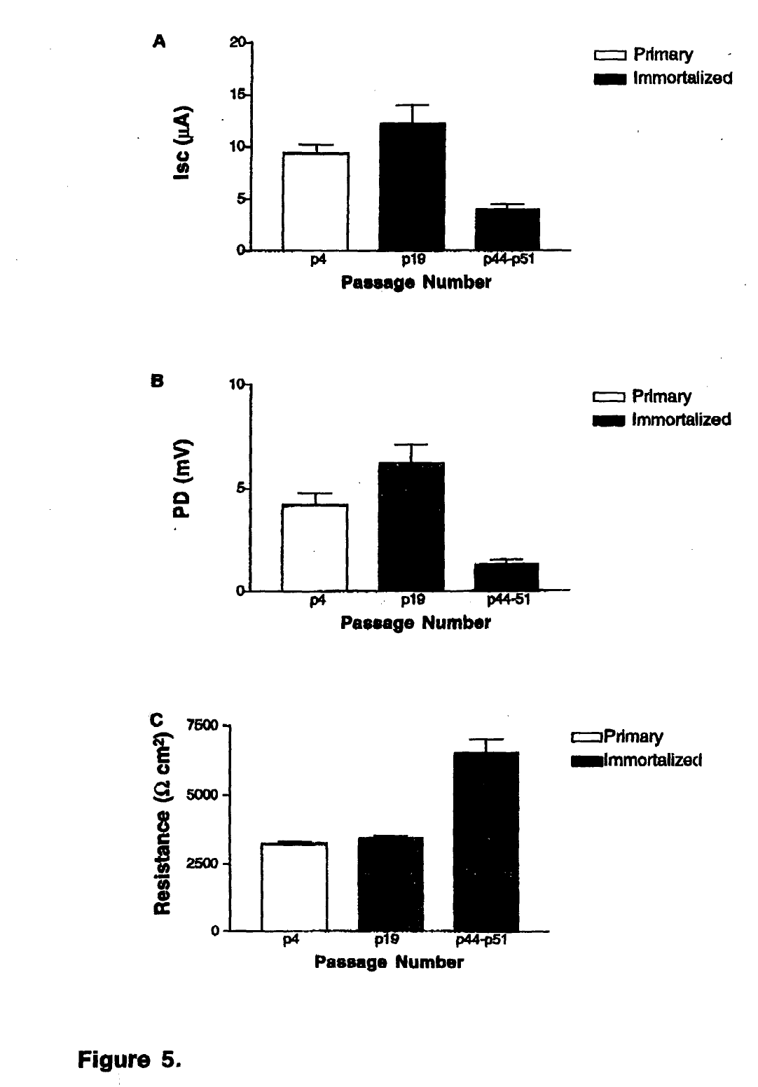

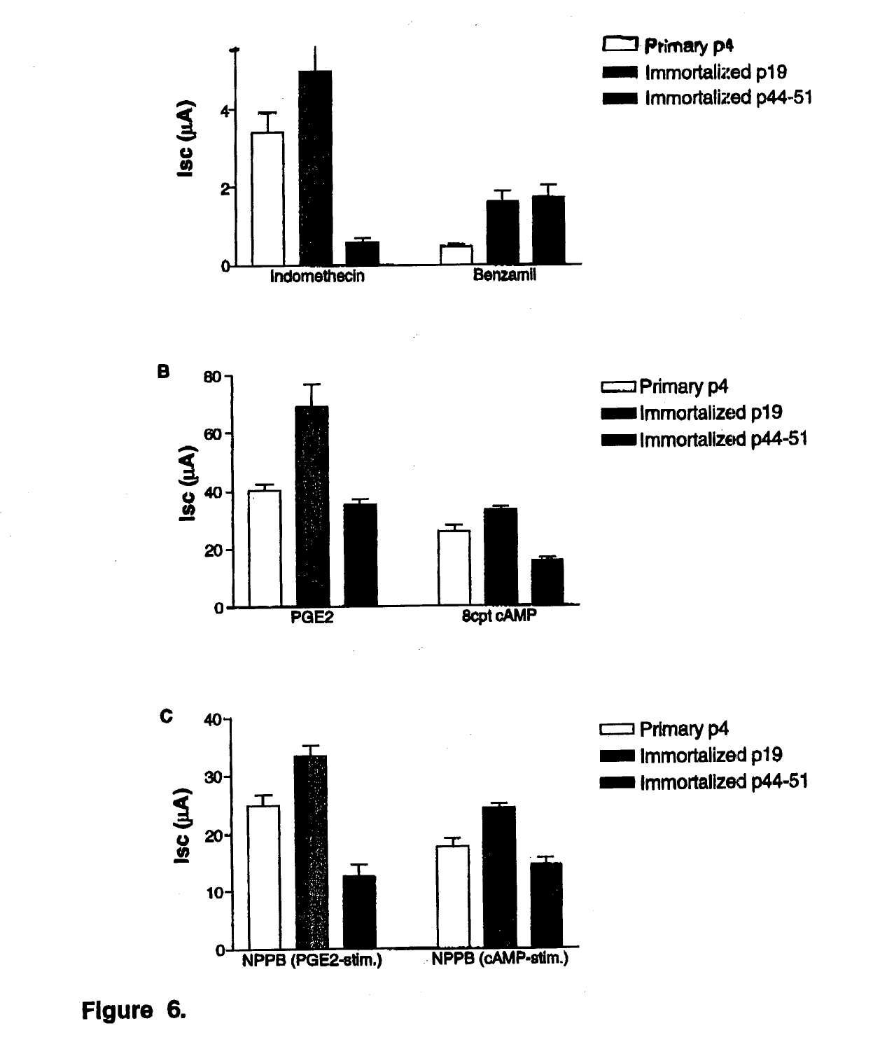

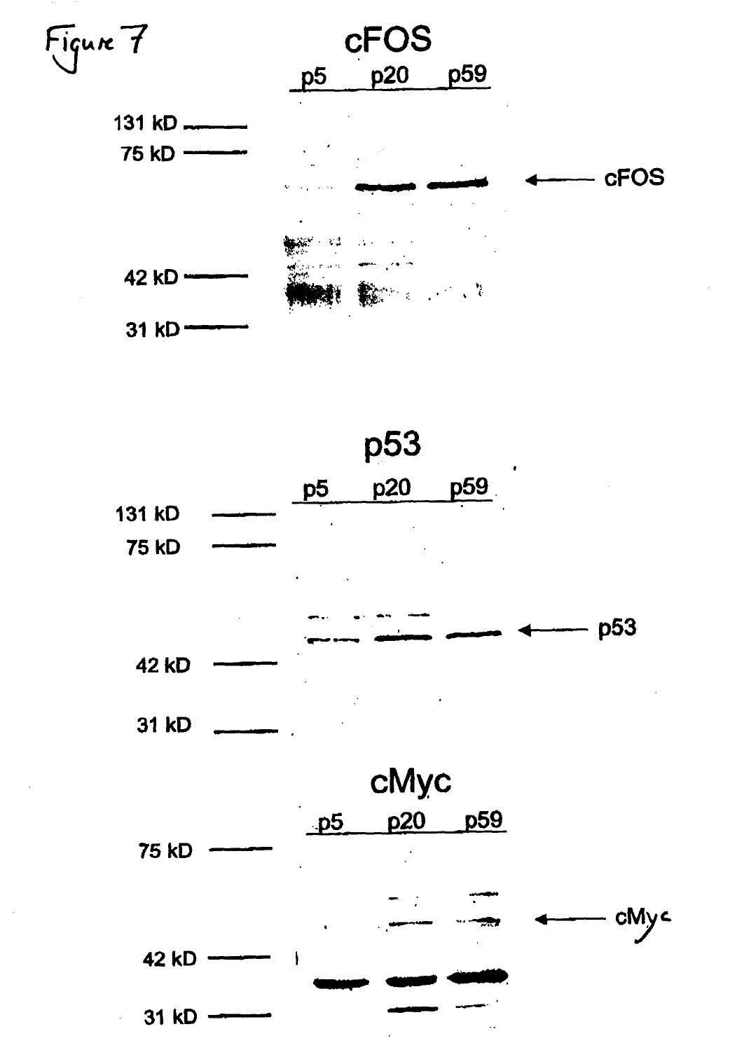

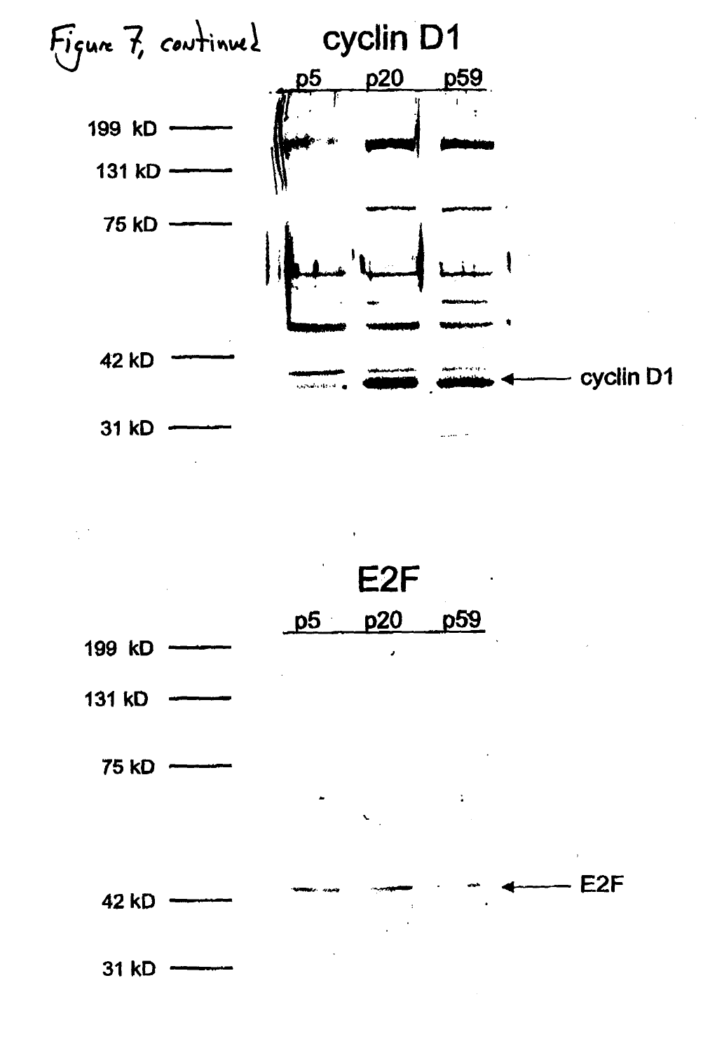

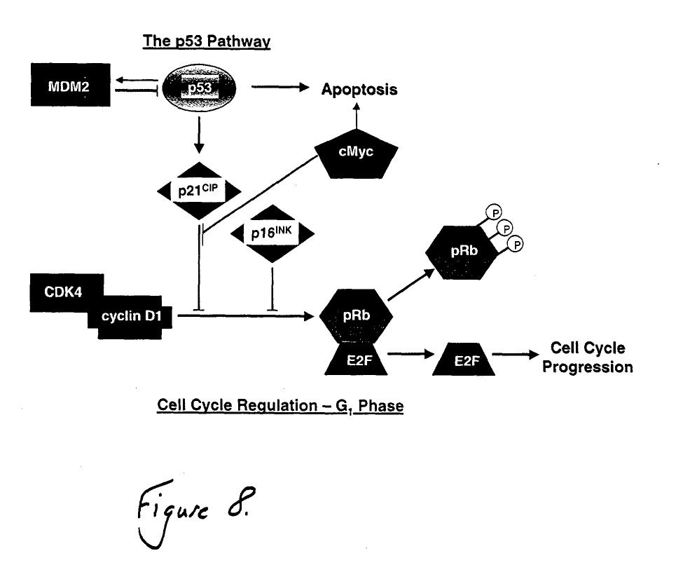

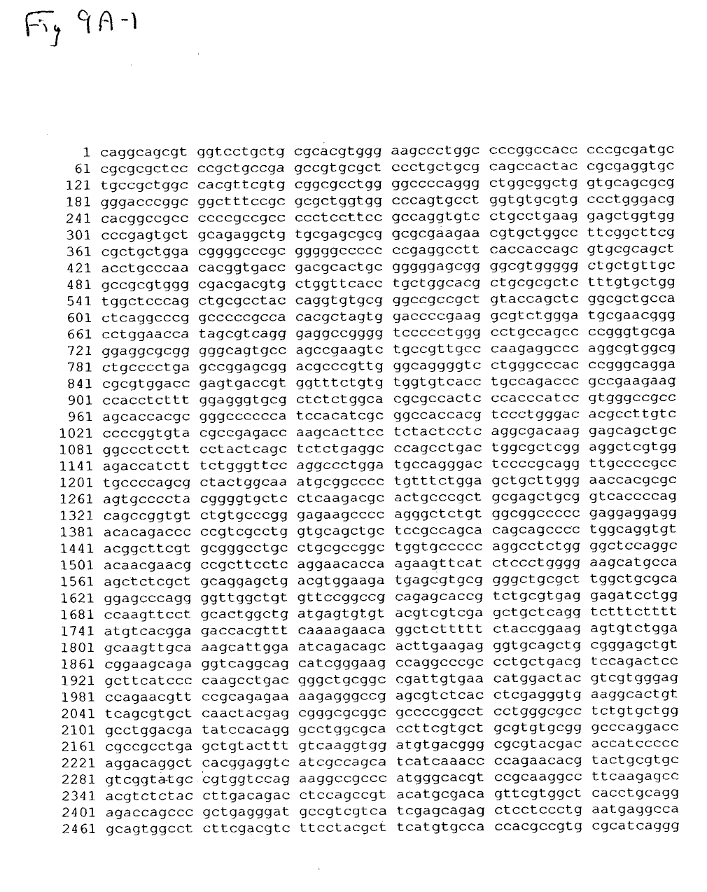

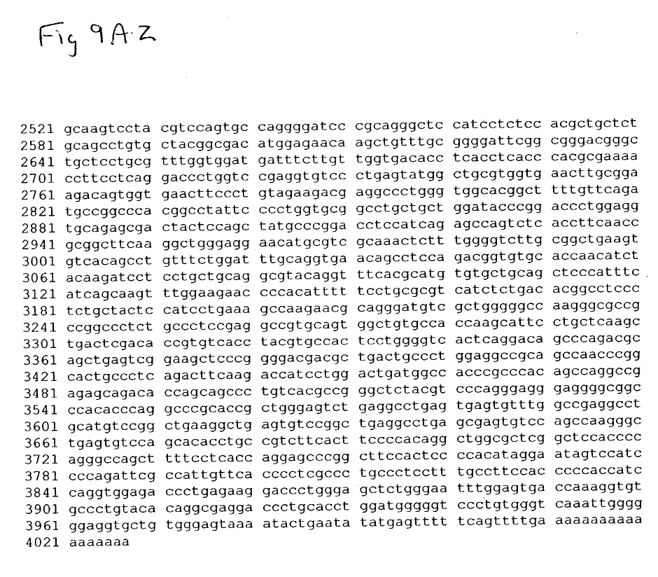

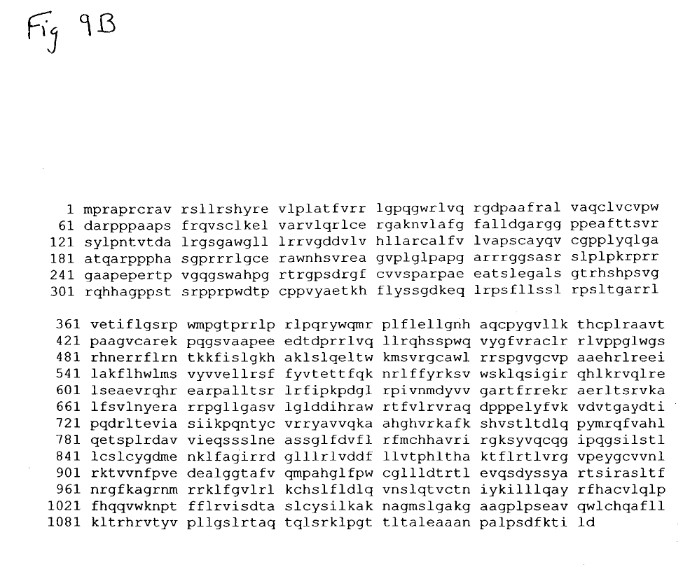

The results are shown in FIGS. 2-8

### CONCLUSION

The results of this study demonstrated that transfection of the human telomerase gene into rapidly proliferating endometrial epithelial cells could produce immortalization. Analysis of the transport phenotype under serum containing conditions showed that early passage immortalized cells (passage 19) exhibited basal transport characteristics that were indistinguishable or enhanced compared to primary cells under the same culture conditions. However, some differences in basal indomethecin-sensitive current and peak responses to PGE2 and 8(4-chlorophenylthiol) cAMP (8 cpt cAMP) were noted in late passage immortalized cells (passage 44-51).

Analysis of key cell cycle regulatory pathways showed that expression of early transcription factors (cFOS) as well as cyclins, such as D1, were upregulated in early and late passage immortalized cells. Unexpectedly, expression of p53, which normally functions to inhibit cell proliferation was also increased in immortalized cells. This unusual result suggested that there may be factors present in immortalized cells that block the inhibitory actions of p53.

The observation that early passage immortalized cells retain a primary epithelial cell transport phenotype indicates that these cells will be useful in future studies directed towards understanding mechanisms and regulation of ion transport across the endometrial epithelium, as well as future studies requiring primary epithelial cell characteristics.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. An immortal porcine cell comprising a polynucleotide encoding an exogenous telomerase reverse transcriptase polypeptide, wherein a complement of the polynucleotide hybridizes to nucleotides 57 to 3455 of SEQ ID NO:1 in a solution containing 2×SSC and 50% formamide at 42° C. for at least about 18 hours, followed by 3 washes for about 30 minutes each at about 50° C. to about 65° C. in a solution containing 0.1×SSC and 0.1% SDS.

2. The immortal porcine cell of claim 1 wherein the polynucleotide is isolated from a human.

3. The immortal porcine cell of claim 1 wherein the polynucleotide comprises nucleotides 57 to 3455 of SEQ ID NO:1.

4. The immortal porcine cell of claim 1 wherein the polynucleotide encodes a polypeptide of SEQ ID NO:2.

5. The immortal porcine cell of claim 1 wherein the polynucleotide is integrated into the cell genome.

6. The immortal porcine cell of claim 1 wherein the cell is diploid.

7. The immortal porcine cell of claim 1 wherein the cell is aneuploid.

8. The immortal porcine cell of claim 1 wherein the cell is obtained from uterine endometrial glandular tissue.

9. The immortal porcine cell of claim 8 wherein the cell is an epithelial cell.

10. The immortal porcine cell of claim 1 wherein a polypeptide is present in the cell in greater concentration compared to a primary porcine cell incubated under the same conditions, the polypeptide selected from the group consisting of cFOS, cyclin D1, and p53.

11. An immortal porcine cell comprising a polynucleotide encoding an exogenous telomerase reverse transcriptase polypeptide, wherein the polynucleotide has at least about 80% identity to nucleotides 57 to 3455 of SEQ ID NO:1.

12. The immortal porcine cell of claim 11 wherein the polynucleotide is isolated from a human.

13. The immortal porcine cell of claim 11 wherein the polynucleotide comprises nucleotides 57 to 3455 of SEQ ID NO:1.

14. The immortal porcine cell of claim 11 wherein the polynucleotide encodes a polypeptide of SEQ ID NO:2.

15. The immortal porcine cell of claim 11 wherein the polynucleotide is integrated into the cell genome.

16. The immortal porcine cell of claim 11 wherein the cell is diploid.

17. The immortal porcine cell of claim 11 wherein the cell is aneuploid.

18. The immortal porcine cell of claim 11 wherein the cell is obtained from uterine endometrial glandular tissue.

19. The immortal porcine cell of claim 18 wherein the cell is an epithelial cell.

20. The immortal porcine cell of claim 11 wherein a polypeptide is present in the cell in greater concentration compared to a primary porcine cell incubated under the same conditions, the polypeptide selected from the group consisting of cFOS, cyclin D1, and p53.

21. An immortal porcine cell comprising an exogenous telomerase reverse transcriptase polypeptide, wherein the polypeptide has at least about 80% identity to SEQ ID NO:2.

22. The immortal porcine cell of claim 21 wherein the polypeptide comprises SEQ ID NO:2.

23. The immortal porcine cell of claim 21 wherein the cell is diploid.

24. The immortal porcine cell of claim 21 wherein the cell is aneuploid.

25. The immortal porcine cell of claim 21 wherein the cell is obtained from uterine endometrial glandular tissues.

26. The immortal porcine cell of claim 25 wherein the cell is an epithelial cell.

27. The immortal porcine cell of claim 21 wherein a polypeptide is present in the cell in greater concentration compared to a primary porcine cell incubated under the same conditions, the polypeptide selected from the group consisting of cFOS, cyclin D1, and p53.

28. An immortal porcine cell comprising a polynucleotide encoding an exogenous telomerase reverse transcriptase polypeptide comprising SEQ ID NO:2.

29. The immortal porcine cell of claim 28 wherein the polynucleotide is isolated from a human.

30. The immortal porcine cell of claim 28 wherein the polynucleotide is integrated into the cell genome.

31. The immortal porcine cell of claim 28 wherein the cell is diploid.

32. The immortal porcine cell of claim 28 wherein the cell is aneuploid.

33. The immortal porcine cell of claim 28 wherein the cell is obtained from uterine endometrial glandular tissue.

34. The immortal porcine cell of claim 33 wherein the cell is an epithelial cell.

35. The immortal porcine cell of claim 28 wherein a polypeptide is present in the cell in greater concentration compared to a primary porcine cell incubated under the same conditions, the polypeptide selected from the group consisting of cFOS, cyclin D1, and p53.

36. A method for making an immortal cell, the method comprising:
providing an isolated primary porcine cell;
introducing to the cell a polynucleotide encoding an exogenous telomerase reverse transcriptase polypeptide, wherein a complement of the polynucleotide hybridizes to nucleotides 57 to 3455 of SEQ ID NO:1 in a solution containing 2×SSC and 50% formamide at 42° C. for at least about 18 hours, followed by 3 washes for about 30 minutes each at about 55° C. in a solution containing 0.1×SSC and 0.1% SDS; and
identifying a cell comprising the polynucleotide, and replicating the cell comprising the polynucleotide in culture at least about 65 passages.

37. The method of claim 36 wherein the polynucleotide comprises nucleotides 57 to 3455 of SEQ ID NO:1.

38. The method of claim 36 wherein the polynucleotide encodes a polypeptide of SEQ ID NO:2.

39. The method of claim 36 wherein the primary cell is obtained from uterine endometrial glandular tissue.

40. The method of claim 39 wherein the primary cell is an epithelial cell.

41. A method for measuring an amount of a virus in a sample, the method comprising:
providing a serial dilution of sample comprising a virus;
contacting an immortal porcine cell comprising an exogenous telomerase reverse transcriptase polypeptide with a portion of the serial dilution; and
measuring the amount of virus present in the virus dilution.

42. The method of claim 41 wherein the virus is a porcine reproductive and respiratory syndrome virus.

43. A method for producing virus, the method comprising:
providing an immortal porcine cell comprising an exogenous telomerase reverse transcriptase polypeptide;
introducing a virus to the immortal porcine cell;
incubating the immortal porcine cell; and
harvesting the virus produced by the immortalized porcine cell.

44. The method of claim 43 wherein the virus is a porcine reproductive and respiratory syndrome virus.

45. A method of evaluating toxicity of a compound, the method comprising:
contacting an immortal porcine cell comprising an exogenous telomerase reverse transcriptase polypeptide with a compound; and
incubating the immortal porcine cell, wherein an increase in doubling time of the immortal porcine cell indicates the compound is toxic.
